(19) United States
(12) Patent Application Publication
(10) Pub. No.: US 2006/0019288 A1
(43) Pub. Date: Jan. 26, 2006

(54) Methods, reaction mixtures, and kits for ligating polynucleotides

(75) Inventors: Mark R. Andersen, Calrlsbad, CA (US); H. Michael Wenz, Redwood City, CA (US); Caifu Chen, Palo Alto, CA (US); Achim Karger, Foster City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/173,112

(22) Filed: Jun. 30, 2005

Related U.S. Application Data
(60) Provisional application No. 60/584,682, filed on Jun. 30, 2004.

(51) Int. Cl. C12Q 1/68; C12P 19/34

(52) U.S. Cl. 435/6; 435/91.2

(57) ABSTRACT

The present teachings pertain to methods, reaction mixtures, and kits for ligating polynucleotides. In some embodiments, a heat-activatable ligation agent, a phosphorylation agent, and a decontamination agent are included in the same ligation reaction mixture with at least one probe set, at least one linker set, and at least one target polynucleotide. A reaction at a first temperature results in hybridization of the probes to the target, phosphorylation of the probes, and decontamination of unwanted reaction components. A reaction at a second temperature results in the ligation of the probes together. In some embodiments, the present teachings are applied in highly multi-plexed ligation reactions in which a plurality of single nucleotide polymorphisms in a plurality of target polynucleotides are queried, and eventually detected using a mobility dependent analysis technique.

### CROSS REFERENCE TO RELATED APPLICATIONS

This application claims priority benefit under 35 U.S.C. § 119(e) from U.S. Provisional Patent Application No. 60/584,682, filed Jun. 30, 2004, which is incorporated herein by reference.

### FIELD

The present teachings generally relate to methods, kits, and reaction mixtures for ligating polynucleotides. The teachings also relate to ligation-based methods, kits, and compositions for determining polynucleotide sequences, including determining single nucleotide polymorphisms in highly multiplexed reactions.

### BACKGROUND

The detection of the presence or absence of (or quantity of) one or more target polynucleotides in a sample or samples containing one or more target sequences is commonly practiced. For example, the detection of cancer and many infectious diseases, such as AIDS and hepatitis, routinely includes screening biological samples for the presence or absence of diagnostic nucleic acid sequences. Also, detecting the presence or absence of nucleic acid sequences is often used in forensic science, paternity testing, genetic counseling, and organ transplantation.

An organism's genetic makeup is determined by the genes contained within the genome of that organism. Genes are composed of long strands or deoxyribonucleic acid (DNA) polymers that encode the information needed to make proteins. Properties, capabilities, and traits of an organism often are related to the types and amounts of proteins that are, or are not, being produced by that organism.

A protein can be produced from a gene as follows. First, the information that represents the DNA of the gene that encodes a protein, for example, protein “X”, is converted into ribonucleic acid (RNA) by a process known as “transcription.” During transcription, a single-stranded complementary RNA copy of the gene is made. Next, this RNA copy, referred to as protein X messenger RNA (mRNA), is used by the cell's biochemical machinery to make protein X, a process referred to as “translation.” Basically, the cell's protein manufacturing machinery binds to the mRNA, “reads” the RNA code, and “translates” it into the amino acid sequence of protein X. In summary, DNA is transcribed to make mRNA, which is translated to make proteins.

The amount of protein X that is produced by a cell often is largely dependent on the amount of protein X mRNA that is present within the cell. The amount of protein X mRNA within a cell is due, at least in part, to the degree to which gene X is expressed. Whether a particular gene or gene variant present, and if so, with how many copies, can have significant impact on an organism. Whether a particular gene or gene variant is expressed, and if so, to what level, can have a significant impact on the organism.

Techniques that can measure the presence of gene targets in a rapid, economical manner with high-throughput and high accuracy are needed in the art. Rapidity can be achieved in a number of ways, including for example reducing the number of different sample handling steps as well as performing more than one manipulation in the same reaction mixture.

### SUMMARY

In some embodiments, the present teachings provide a method for reducing the number of workflow steps in a ligation reaction comprising, providing a target polynucleotide sequence, a heat-activatable ligase, a first probe, a second probe, a phosphorylation agent, and a decontamination agent, thereby forming a reaction mixture. Then, performing a phosphorylation reaction comprising the phosphorylation agent at a first temperature and performing a decontamination reaction comprising the decontamination agent at the first temperature, wherein the ligase is substantially inactive at the first temperature. Then, increasing the reaction temperature to a second temperature thereby increasing the activity of the ligase and performing a ligation reaction wherein the first probe is ligated to the second probe, thereby reducing the number of processing steps in a ligation reaction as compared with a ligation reaction in which the phosphorylation reaction and decontamination reaction are performed in reactions separate from the ligation reaction.

Some embodiments of the present teachings provide a reaction mixture comprising a heat-activatable ligase, a phosphorylation agent, a decontamination agent, a target polynucleotide, a first probe, and a second probe.

Some embodiments of the present teachings provide a kit comprising a ligation master mix and at least one probe set, wherein the ligation master mix comprises at least one heat-activatable ligase, at least one phosphorylation agent, at least one decontamination agent, and at least one buffer.

### BRIEF DESCRIPTION OF THE DRAWINGS

FIG. 1 provides a schematic of some work-flow characteristics according to the present teachings wherein a plurality of different reactions are performed in separate reaction vessels.

FIG. 2 provides a schematic of some work-flow characteristics according to the present teachings wherein a plurality of different reactions are performed in the same reaction vessel.

FIG. 3 provides a schematic of the reaction steps according to Example 1 of the present teachings. Here, the identity of a single nucleotide polymorphism is queried. The first probe one and first probe two are indicated as ASOa1 and ASOa2 (for allele-specific oligonucleotide 1 and allele-specific oligonucleotide 2), the second probe is indicated as LSO (for locus specific oligo), and the mobility probe is referred to as a Zipchute probe.

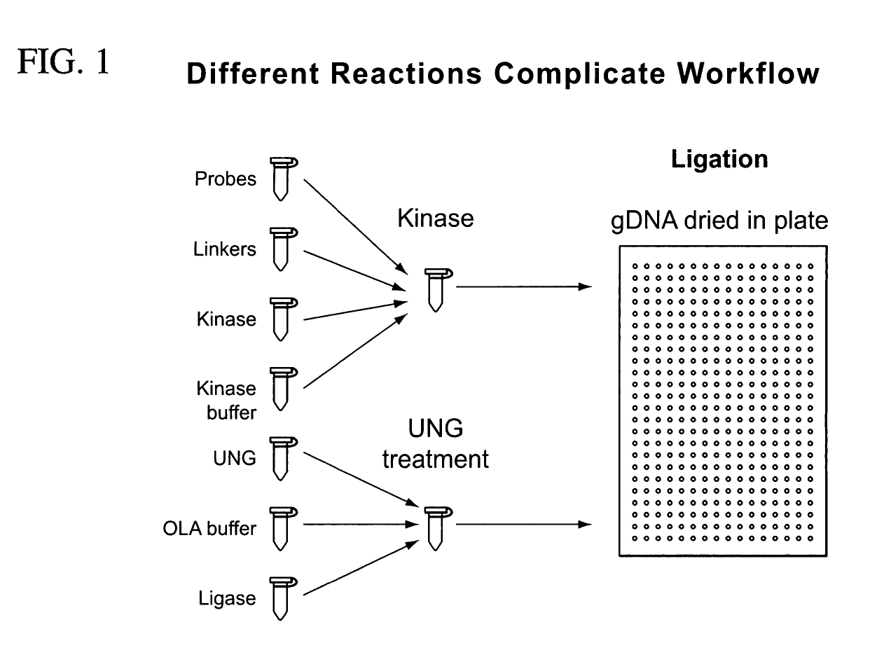

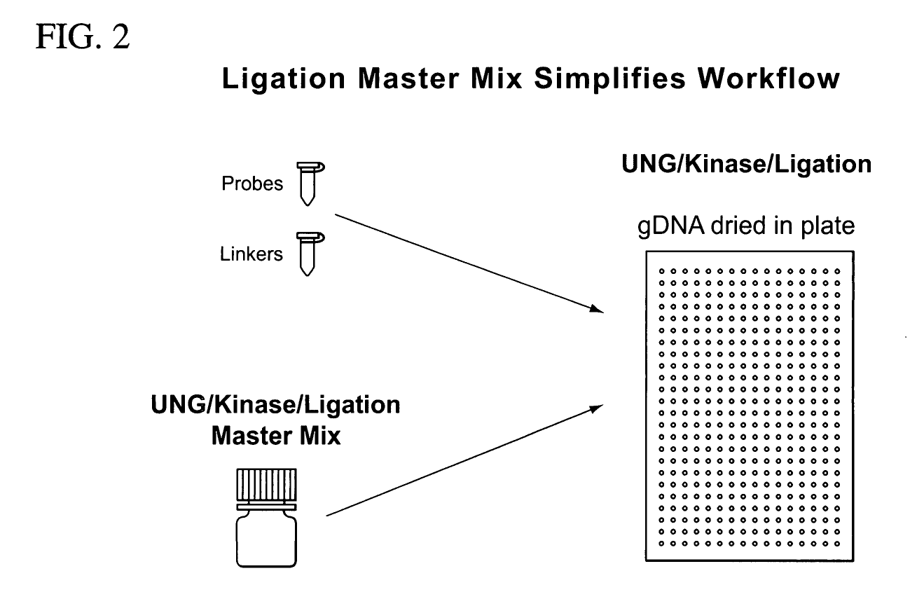

### DESCRIPTION OF EXEMPLARY EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control.

It is to be understood that both the foregoing general description and the following detailed description are exemplary and explanatory only and are not restrictive of the invention. In this application, the use of the singular includes the plural unless the context specifically dictates otherwise. For example, “a probe” means that more than one probe can be present; for example, one or more copies of a particular probe species, as well as one or more versions of a particular probe type. Also, the use of “or” means “and/or” unless stated otherwise. Similarly, “comprise”, “comprises”, “comprising”, “include”, “includes”, and “including” are not intended to be limiting.

Definitions

As used herein, the term “heat-activatable ligase” refers to a ligase that is substantially inactive at lower temperatures and requires higher temperatures for activation. Typically, heat-activatable ligases are substantially inactive at around room temperature (25C), and can become activated at higher temperatures.

As used herein, the term “first probe” refers to the probe in a ligation reaction that provides the free 3′ end that is ligated to the 5′ end of a contiguously hybridized second probe.

As used herein, the term “second probe” refers to the probe in a ligation reaction that provides the free 5′ end that is ligated to the 3′ end of a contiguously hybridized first probe.

As used herein, the term “phosphorylation agent” refers to an agent that can add a phosphate group to a probe. Typically a phosphorylation agent is a polynucleotide kinase.

As used herein, the term “decontamination agent” refers to an agent that can remove contaminating reaction components from a reaction. Typically, a decontamination agent is a uracil-N-glycosylase (UNG) or a Uracil-DNA Glycosylase—(UDG) and the contaminating reaction components comprise uracil, thus rendering them susceptible to degradation by UNG or UDG.

As used herein, the term “ligation agent” refers to an agent that can ligate two probes together in a ligation reaction. Typically, a ligation agent is a ligase enzyme, although according to the present teachings can comprise any number of enzymatic or non-enzymatic reagents. For example, a ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits.

As used herein, the term “probe set” refers to at least one first probe and at least one second probe that can hybridize to and query a target polynucleotide sequence. In multiplexed reactions, a plurality of probe sets are employed to query a plurality of target polynucleotides.

As used herein, the term “linker set” refers to polynucleotides that can ligate to the probes in a probe set and introduce spacers and sequence information that can be subsequently detected.

As used herein, the term “target polynucleotide” refers to a region or subsequence of a nucleic acid that can be queried.

As used herein, the term “nucleic acid” refers to both naturally-occurring molecules such as DNA and RNA, but also various derivatives and analogs. Generally, the probes, linkers, and target polynucleotides of the present teachings are nucleic acids, and typically comprise DNA. Additional derivatives and analogs can be employed as will be appreciated by one having ordinary skill in the art. For example universal nucleotides can include, but are not limited to, deoxy-7-azaindole triphosphate (d7AITP), deoxyisocarbostyril triphosphate (dlCSTP), deoxypropynylisocarbostyril triphosphate (dPICSTP), deoxymethyl-7-azaindole triphosphate (dM7AITP), deoxylmPy triphosphate (dlmPyTP), deoxyPP triphosphate (dPPTP), or deoxypropynyl-7-azaindole triphosphate (dP7AITP). Additional illustrative examples can be found regarding universal bases in Loakes, N.A.R. 2001, vol 29:2437-2447, Seela N.A.R. 2000, vol 28:3224-3232, Published U.S. application Ser. No. 10/290,672, and U.S. Pat. No. 6,433,134. Illustrative teaching regarding locked nucleic acids can be found in Published P.C.T. Application WO 98/22489; Published P.C.T. Application WO 98/39352; and Published P.C.T. Application WO 99/14226. Further, sugars can include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides can include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). In some embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions. Other nucleic acid analogs and bases include for example intercalating nucleic acids (INAs, as described in Christensen and Pedersen, 2002), and AEGIS bases (Eragen, U.S. Pat. No. 5,432,272). Additional descriptions of various nucleic acid analogs can also be found for example in (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048. Other nucleic analogs comprise phosphorodithioates (Briu et al., J. Am. Chem. Soc. 11 1:2321 (1989), O-methylphophoroamidite linkages (see Eckstein, Oligonucleotides and Analogues: A Practical Approach, Oxford University Press), those with positive backbones (Denpcy et al., Proc. Natl. Acad. Sci. USA 92:6097 (1995); non-ionic backbones (U.S. Pat. Nos. 5,386,023, 5,386,023, 5,637,684, 5,602,240, 5,216,141, and 4,469,863. Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (194): Chaq.ters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biornolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) ppl69-176). Several nucleic acid analogs are also described in Rawls, C & E News Jun. 2, 1997 page 35. Unless denoted otherwise, whenever a nucleic acid sequence is represented, it will be understood that the nucleotides are in 5′ to 3′ order from left to right and that “A” denotes deoxyadenosine, “C” denotes deoxycytosine, “G” denotes deoxyguanosine, and “T” denotes thymidine, unless otherwise noted. Additionally, the “nucleic acids” of the present teachings can also comprise “peptide nucleic acid” or “PNA,” including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, and 6,107,470. The term PNA also applies to any oligomer or polymer segment comprising two or more subunits of those nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Teft. Left. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et J. Chem. Soc. Perkin Trans. 11: 547-554 (1997); Lowe et al., J. Chem. Soc. Perkin Trans. 11:555-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapefti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000. A PNA can also be an oligomer or polymer segment comprising two or more covalently linked subunits of the formula found in paragraph 76 of U.S. Patent Application 2003/0077608A1.

As used herein, “amplification” refers to any means by which at least a part of at least one target polynucleotide, ligation product, at least one ligation product surrogate, or combinations thereof, is reproduced, typically in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook and Russell; Sambrook et al.; Ausbel et al.; PCR Primer: A Laboratory Manual, Diffenbach, Ed., Cold Spring Harbor Press (1995); The Electronic Protocol Book, Chang Bioscience (2002)(“The Electronic Protocol Book”); Msuih et al., J. Clin. Micro. 34:501-07 (1996); The Nucleic Acid Protocols Handbook, R. Rapley, ed., Humana Press, Totowa, N.J. (2002)(“Rapley”); Abramson et al., Curr Opin Biotechnol. 1993 February;4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February;12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1.

In some embodiments, amplification comprises at least one cycle of the sequential procedures of: hybridizing at least one primer with complementary or substantially complementary sequences in at least one ligation product, at least one ligation product surrogate, or combinations thereof; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally. In some embodiments, newly-formed nucleic acid duplexes are not initially denatured, but are used in their double-stranded form in one or more subsequent steps.

Primer extension is an amplifying means that comprises elongating at least one probe or at least one primer that is annealed to a template in the 5′ to 3′ direction using an amplifying means such as a polymerase. According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed probe or primer, to generate a complementary strand. In some embodiments, primer extension can be used to fill a gap between two probes of a probe set that are hybridized to target sequences of at least one target nucleic acid sequence so that the two probes can be ligated together. In some embodiments, the polymerase used for primer extension lacks or substantially lacks 5′ exonuclease activity.

In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carryover of previous uracil-containing products by the use of uracil-N-glycosylase (see for example Published P.C.T. Application WO9201814A2). In some embodiments of the present teachings, any of a variety of techniques can be employed prior to amplification in order to facilitate amplification success, as described for example in Radstrom et al., Mol Biotechnol. 2004 February; 26(2):133-46. In some embodiments, amplification can be achieved in a self-contained integrated approach comprising sample preparation and detection, as described for example in U.S. Pat. Nos. 6,153,425 and 6,649,378.

Detection

It will be appreciated that the detection, if any, of the ligation product or ligation product surrogate is not a limitation of the present teachings. Detection can be achieved in some embodiments by employing a donor moiety and signal moiety, and one can use certain energy-transfer fluorescent dyes for detection of the ligation product. Certain non-limiting exemplary pairs of donors (donor moieties) and acceptors (signal moieties) are illustrated, e.g., in U.S. Pat. Nos. 5,863,727; 5,800,996; and 5,945,526. Use of some such combinations of a donor and an acceptor have also been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, Vic™, LiZ™, Tamra™, 5-Fam™, 6-Fam™, and Texas Red (Molecular Probes). (Vic™, LiZ™, Tamra™, 5-Fam™, and 6-Fam™ (all available from Applied Biosystems, Foster City, Calif.) In some embodiments, the amount of signaling probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator. According to some embodiments, one can employ an internal standard to quantify the amplification product indicated by the fluorescent signal. See, e.g., U.S. Pat. No. 5,736,333.

In some embodiments amplified ligation products may be measured with DNA binding dyes such as ethidium bromide of SYBR green 1 dye.

Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), and the ABI GeneAmp® 7500 Sequence Detection System (Applied Biosystems, Foster City, Calif.).

In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product.

In some embodiments, combined thermal cycling and fluorescence detecting devices can be used for precise quantification of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification.

According to some embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target polynucleotide.

According to some embodiments, the amplification products can be scored as positive or negative as soon as a given number of cycles is complete. In some embodiments, the results may be transmitted electronically directly to a database and tabulated. Thus, in some embodiments, large numbers of samples may be processed and analyzed with less time and labor required.

According to some embodiments, different signaling probes may distinguish between different target nucleic acid sequences. A non-limiting example of such a probe is a 5′-nuclease fluorescent probe, such as a TaqMan® probe molecule, wherein a fluorescent molecule is attached to a fluorescence-quenching molecule through an oligonucleotide link element. In some embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WL_{A} and WL_{B}) and that are specific to two different identifying portions of two different ligation products (A′ and B′, respectively). Ligation product A′ is formed if target nucleic acid sequence A is in the sample, and ligation product B′ is formed if target nucleic acid sequence B is in the sample. In some embodiments, ligation product A′ and/or B′ may form even if the appropriate target nucleic acid sequence is not in the sample, but such ligation occurs to a measurably lesser extent than when the appropriate target nucleic acid sequence is in the sample. After amplification, one can determine which specific target nucleic acid sequences are present in the sample based on the wavelength of signal detected. Thus, if an appropriate detectable signal value of only wavelength WL_{A} is detected, one would know that the sample includes target nucleic acid sequence A, but not target nucleic acid sequence B. If an appropriate detectable signal value of both wavelengths WL_{A} and WL_{B} are detected, one would know that the sample includes both target nucleic acid sequence A and target nucleic acid sequence B.

In some embodiments, melting curve analysis may be used to distinguish between different target nucleic acid sequences

In some embodiments, ligation products or ligation product surrogates can be detected by a mobility-dependent analysis technique, including analytical techniques based on differential rates of migration between different analyte species. Exemplary mobility-dependent analysis techniques include electrophoresis, chromatography, mass spectroscopy, sedimentation, e.g., gradient centrifugation, field-flow fractionation, multi-stage extraction techniques, and the like.

In some embodiments, detection of the ligation product can be achieved with capillary electrophoresis. For example, probes in the ligation reaction can comprise identifying portions, and following amplification mobility probes comprising a sequence complementary to the identifying portion can be hybridized to the amplification product. After removing unhybridized mobility probes, the bound mobility probes can be eluted and detected with a mobility dependent analysis technique such as capillary electrophoresis. For illustrative teachings in capillary electorphoresis detection and mobility probes, see for example U.S. Pat. Nos. 5,777,096, 6,624,800, 5,470,705, 5,514,543, and 6,395,486.

Aspects of the present teachings may be further understood in light of the following examples, which should not be construed as limiting the scope of the teachings in any way.

### Exemplary Embodiments

The present teachings can find application in a variety of contexts. For example, the present teachings can be applied in highly multiplexed ligation reactions comprising probes designed to query a plurality of target polynucleotides. In some embodiments, a plurality of multiplexed reactions are performed in a microtitre plate. When working with microtitre plates, setting up the large number of separate multiplexed reactions can be very time-intensive. For example, it can be time-intensive to deliver reaction components (enzymes, probes, buffer, etc) to each well of a 96 well microtitre dish. It can be more time-intensive in situation involving a plurality of microtitre dishes. It will be appreciated that the situation can be exacerbated when 384-well microtitre plates are involved. It will further be appreciated that the situation can be exacerbated in complex reaction involving a variety of chemical manipulations such as phosphorylation, degradation of unwanted reaction contaminants, polymerase extension, and ligation.

In conventional approaches, such time-intensive reaction set-ups can result in premature, and often-times non-specific, probe ligation Some embodiments of the present teachings provide methods for reducing the amount of non-specific ligation in multiplexed ligation reactions. In some embodiments, the reduction in non-specific ligation is achieved by using unphosphorylated (unligatable) probes, or by providing a heat-activatable ligase. Heat-activatable ligases can have the property of being substantially inactive at lower temperatures, and require temperature elevation in order to provide for substantial activity. In some embodiments, the multiplexed reaction set-up can occur at a lower temperature (for example, room temperature, or on ice). Following the set up, the reaction temperature can be elevated to provide for substantial activity of the ligase. In some embodiments, a reduction in processing steps can be achieved by providing additional enzymes in the ligation reaction mixture, as will be described further infra. In some embodiments, heat-activatable enzymes (for example, heat-activatable ligases), can be used along with additional enzymes, as will become more clear infra.

For example, some embodiments of the present teachings provide for ligation reactions comprising a heat-activatable ligase and at least one additional enzyme. Some embodiments of the present teachings provide methods for ligating polynucleotides together in a single reaction mixture comprising, removing unwanted contaminants by a decontamination agent such as uracil-N-glycosylase, phosphorylating probes by a phosphorylating agent such as a kinase, and ligating probes together using a ligation agent such as a ligase, wherein the ligase is substantially inactive at a first temperature during which the phosphorylation agent and decontamination agent are active, and the ligase is substantially active at a second temperature. In some embodiments, the phosphorylation agent, and/or decontamination agent are inactivated at the second temperature. In some embodiments, a reaction can comprise a heat-activatable ligation agent and a phosphorylation agent, but no decontamination agent. In some embodiments, a reaction can comprise a heat-activatable ligation agent a decontamination agent, but no phosphorylation agent.

It will be appreciated that a variety of heat-activatable strategies can be employed in the context of the present teachings. For example, a heat-activatable phosphorylation agent could be employed in a ligation reaction further comprising a ligation agent and a decontamination reagent. In such a scenario, the probes can initially lack 5′ phosphate groups. As a result, no ligation could occur until the temperature is reached that allows for the activation of the phosphorylation agent, and hence, phosphorylation of the probes.

It will also be appreciated that a variety of strategies can used to make an enzyme heat-activatable, that such procedures are routine in contemporary molecular biology laboratories, and that their implementation in no way requires undue experimentation. Representative teachings on various approaches for making heat-activatable enzymes are available and include antibody approaches (see for example U.S. Pat. No. 5,338,671), chemical approaches including for example citraconic anhydride (see for example U.S. Pat. No. 5,773,258 and U.S. Pat. No. 5,677,152) chemical approaches including aldehydes, such as formaldehyde (see for example U.S. Pat. No. 6,183,998), and aptamer-based approaches (see for example U.S. Pat. No. 6,183,967). Additional methods for producing heat-activatable enzymes involve mineral heat-activatable approaches comprising precipitates (see for example Published U.S. Patent Application 20030082567A1), and wax (see Sambrook et al., Molecular Cloning, Third Edition). It will be appreciated that the manner in which an agent (for example an enzyme) is modified to implement the heat-activatable property is not a limitation of the present teachings.

Some embodiments of the present teachings provide methods for reducing the number of different reagent processing steps in a ligation reaction wherein the ligase is not a heat-activatable ligase. For example, some embodiments of the present teaching comprise ligating polynucleotides together in a single reaction mixture comprising, removing unwanted contaminants by a decontamination agent such as uracil-N-glycosylase, phosphorylating probes by a phosphorylating agent such as a kinase, and ligating probes together using a ligation agent such as a non heat-activatable ligase. In some embodiments, a reaction can comprise a ligation agent and a phosphorylation agent, and no decontamination agent. In some embodiments, a reaction can comprise a ligation agent a decontamination agent, and no phosphorylation agent.

It will be appreciated that any of a variety of decontamination agents can be employed in the context of the present teachings, though typically uracil-N-glycosylases are used. A number of uracil-N-glycosylases are available, for example those collected from gram-positive microorganisms such as e.g. Arthrobacter or Micrococcus, as described for example in U.S. Pat. No. 6,187,575, and commercially available from Roche as AmpErase. Other examples of glycosylases that can be employed in the present teachings include uracil-DNA glycosylase isolated from E. Coli, and commercially available from New England Biolabs as UDG (and see for example Lindahl, T. et al. (1977) J. Biol. Chem., 252, 3286-3294). In general, it will be appreciated that the kind of uracil-N-glycosylase, or decontamination agent generally, is not a limitation of the present teachings.

In some embodiments, the contaminating reaction components are products from a previously performed ligation reaction wherein U-containing probes were not substrates for UNG prior to ligation.

In some embodiments, a heat-activatable UNG or UDG is contemplated. For example, a non-heat-activatable ligase can be present in a reaction mixture along with a heat-activatable UNG. With probes comprising uracil in appropriate locations, the elevation of reaction temperature to activate the UNG can result in cleavage of the uracils, and thus freeing of a free-phosphate groups on the probes on which the ligase can then act. For example, uracil can be on the 5′ end of first probes, and their cleaveage can result in a ligation-competent complex. Also, flaps comprising uracil can be cleaved to result in ligation-competent complexes.

It will be appreciated that any of a variety of phosphorylation agents can be employed in the context of the present teachings, though typically polynucleotide kinases are used. For example, polynucleotide kinases are commercially available from a variety of sources, including New England Biolabs and Amersham. Additionally, polynucleotide kinases with improved uniform phosphorylation of oligonucleotides independent of the base at the 5′-end, as well as polynucleotide kinases that provide higher labeling (see for example OptiKinase from Amersham Biosciences), can also be employed according to the present teachings. In general, it will be appreciated that the kind of polynucleotide kinase, or phosphorylation agent generally, is not a limitation of the present teachings. It will also be appreciated that according to the present teachings phosphorylation is a biochemical reaction resulting in the addition of a phosphate group to the 5′ end of a polynucleotide, thus rendering it suitable for ligation to a 3′ OH group of a corresponding polynucleotide.

It will be appreciated that the present teachings can be applied in a variety of contexts in which ligation reactions are employed to query the identity of target polynucleotide sequences. For example, various OLA strategies, (see for example Whiteley et al., U.S. Pat. No. 6,054,266, U.S. Pat. No. 5,962,222, U.S. Pat. No. 5,521,065 U.S. Pat. No. 5,242,794, U.S. Pat. No. 4,883,750), FEN-LCR (see for example Bi et al., U.S. Pat. No. 6,511,810, padlock probes (see for example Landegren et al., U.S. Pat. No. 5,871,921), coupled ligation and amplification methods (for example Eggerding et al., U.S. Pat. No. 6,130,073 and U.S. Pat. No. 5,912,148) gap-versions of OLA, LDR, LCR, and such strategies generally known to one having ordinary skill in the art (see Cao et al., 2004, Trends in Biotechnology, Vol. 22, No. 1) for a recent review.

The present teachings contemplate embodiments in which the first probe and second probe are not only different molecules, but also embodiments in which the first probe and the second probe are part of the same molecule (for example, Molecular Inversion Probes commercially available from ParAllele, and U.S. Pat. No. 5,871,921.

It will be appreciated that current teachings can be employed in the context of various linker ligation strategies, as discussed for example in U.S. Non-Provisional application Ser. No. 10/982,619, and the SNPlex™ System User Manuel commercially available from Applied Biosystems. Such strategies can employ concatameric ligation of several probes on a target polynucleotide sequence. These and other strategies (for example see U.S. Pat. No. 6,027,889) can also be employed to allow for various approaches to remove unincorporated reaction components by nuclease-mediated digestion.

It will be appreciated that the present teachings can be employed in the context of various positive and negative control ligation reactions comprising known monomorphic target polynucleotides, thereby allowing for the determination of ligation efficiency, as described for example in U.S. Provisional Patent Application 60/584, 873 to Wenz et al., and co-filed U.S. Non-Provisional Patent Application claiming priority thereto.

In some embodiments, the master mix for the ligation reaction comprises:
20 mM Tris-HCl pH 7.6 at 25C
7 mM MgCl2
20 mM KCl
0.10% Triton X-100
1 mM DTT
1 mM NAD
2.5 units/ul heat-activatable ligase
2 units/ul T4 polynucleotide kinase
0.01 units/ul uracil-N-glycosylase
0.05 mM Desferal
1 mM dATP
5% PEG 8000
   In some embodiments, DTT can be used. DTT is a (sulfhydryl) reducing agent primarily for stability of the enzymes. In some embodiments, TCEP Tris(2-carboxyethyl)phosphine HCl can be used as a reducing agent (0.1-2 mM), which can be more effective, and more stable.
   Exemplary Kits in Accordance with Some Embodiments of the Present Teachings

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In some embodiments, the kit components are optimized to operate in conjunction with one another.

In some embodiments, a kit for ligating polynucleotides is provided. For illustrative kit configurations contemplated by the present teachings, the reader is invited to consult the SNPleX™ System User Manual, commercially available from Applied Biosystems.

Some embodiments of present teachings provide a kit comprising a ligation master mix and at least one probe set, wherein the ligation master mix comprises at least one heat-activatable ligase, at least one phosphorylation agent, at least one decontamination agent, and at least one buffer. In some embodiments, a kit can further comprise at least one linker set. In some embodiments, the phosphorylation agent is a kinase. In some embodiments, the kinase is T4 polynucleotide kinase. In some embodiments, the decontamintation agent is a uracil-N-glycosylase. In some embodiments, the uracil-N-glycosylase is at least one of Arthrobacter, Micrococcus, E. coli, and combinations thereof. In some embodiments the heat-activatable ligase is at least one of Afu, T4 ligase, E. coli ligase, AK16D ligase, Pfu ligase, and combinations thereof. In some embodiments, the ligase is not a heat-activatable ligase. In some embodiments, the phosphorylation agent, and/or the decontamination agent can be heat-activatable.

In some embodiments, the kit can comprise a polymerase used in, for example, mismatch repair, as illustrated in for example the Molecular Inversion probes commercially available from ParAllele (and see U.S. Pat. No. 5,871,921) In some embodiments, the polymerase can be a heat-activatable polymerase.

### EXAMPLE 1

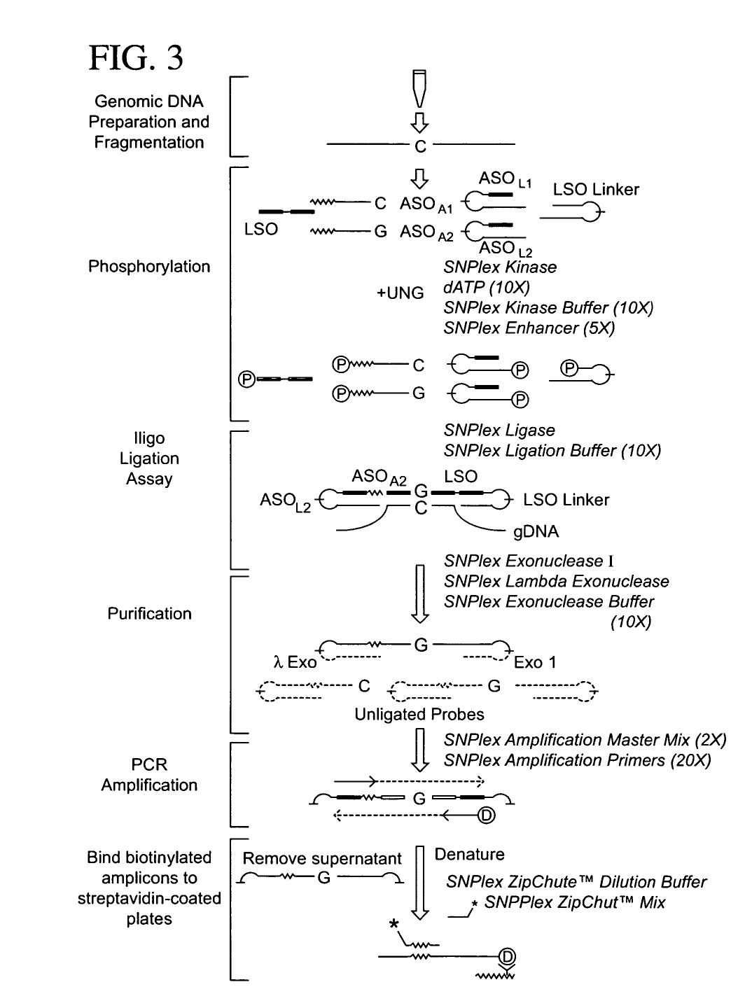

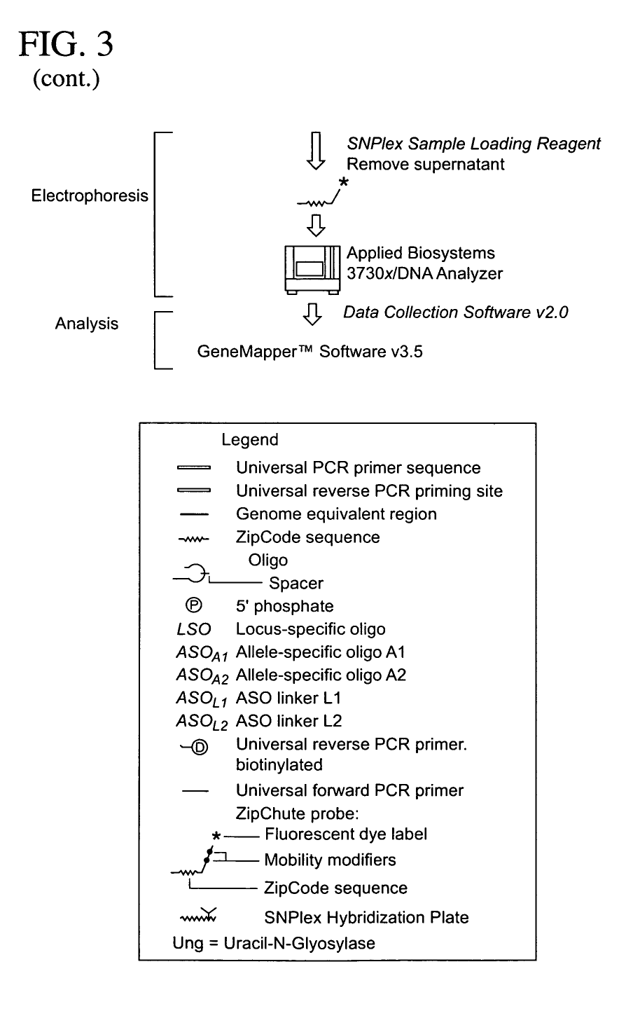

Example 1 provides illustration of the present teachings, wherein a multiplexed ligation reaction is performed with a ligation reaction mixture comprising a heat-activatable ligase, a uracil-N glycosylase, and a T4 polynucleotide kinase. The workflow of this experiment is depicted in FIG. 3. Successful determination of homozygous and heterozygous alleles for a collection of single nucleotide polymorphisms were found for a reaction containing the decontamination agent as compared to a reaction in which the decontamination agent was lacking.

The protocol was basically as follows:

Genomic DNA is fragmented by boiling, quantified, and 37 ng/well was distributed and dried down into 384-well optical plates.

At room temperature, a master mix was pipetted, comprising:
20 mM Tris-HCl ph 7.6 at 25C
7 mM MgCl2
0.10% Triton X-100
1 mM DTT
1 mM NAD
5 units/ul heat-activatable ligase
0.1 units/ul T4 polynucleotide kinase
0.01 units/ul uracil-N-glycosylase (also, a control reaction without UNG was performed)
0.05 mM Desferal
1.25 mM dATP
5% PEG 8000

At room temperature, 0.5 ul of Probes (100 nM each) and Linkers (50 nM of each ASO (allele specific oligonucleotide) linker and 85 nM of each LSO (locus specific oligonucleotide linker) were pipetted into each well of the 384-well optical plate using a Hydra II Plus One robot.

Master mix (4.5 ul per reaction) was pipetted into each well of the 384-well optical plate using a Hydra II Plus One robot.

A ligation reaction was performed on an Applied Biosystems GeneAmp PCR system 9700 with firmware 3.05 with the following cycling conditions:

| Step | Step Type | Temperature (C.) | Time |
|---|---|---|---|
| 1 | Hold | 37 | 60 minutes |
| 2 | Hold | 85 | 30 minutes |
| 2 | 30 cycles | 90 | 15 seconds |
| | | 60 | 30 seconds |
| | | 51 | 30 seconds |
| | | (with 2% ramp) | |
| 3 | Hold | 99 | 10 minutes |
| 4 | Hold | 4 | Hold indefinitely |

An exonuclease clean-up was then performed comprising: For each reaction:
4.2 ul Nuclease-free water
0.5 ul SNPleX™ exonuclease buffer
0.2 ul SNPlex™ lambda exonuclease
0.1 ul SNPlex™ exonuclease 1
5 ul ligation reaction
   at 37C for 90 minutes, followed by 80 C for 10 minutes.

Following the exonuclease clean-up, 10 ul of water was added to each reaction, and a PCR amplification of the ligation products was performed. The PCR was performed in a MicroAmp 384-well reaction plate (Applied Biosystems P/N 4309849) with an ABI Optical Adhesive Cover (P/N 4311971).

First, a 20× universal oligonucleotide primer mixture was formed comprising:
10 uM universal forward primer (UF 19)
10 uM biotinylated universal reverse primer (UR 19)
10 mM Tris HCl, pH 8.0 at 25C
1 mM EDTA

Then, a plurality of 10 ul PCR reactions was set up comprising:
5.0 ul 2× SNPleX™ PCR mix
0.5 ul 20 universal oligonucleotide primer mixture (from above)
2.5 ul H2O
2.0 ul of the nuclease-treated ligation reaction

A PCR reaction was performed on an Applied Biosystems GeneAmp PCR system 9700 with the following cycling conditions:

| Step | Step Type | Temperature (C.) | Time |
|---|---|---|---|
| 1 | Hold | 95 | 10 minutes |
| 2 | 30 cycles | 95 | 15 seconds |
| | | 70 | 60 seconds |
| 3 | Hold | 4 | Hold indefinitely |

Following the PCR, biotinylated strands are captured and separated, and mobility probes are hybridized to the immobilized strands. Eluted mobility probes are then detected via capillary electrophoresis on an Applied Biosystems 3730.

Cluster plots representing the data from a 47-plex experiment performed as described demonstrated the effectiveness of the method (plots not shown).

While the present teachings have been described in terms of these exemplary embodiments, the skilled artisan will readily understand that numerous variations and modifications of these exemplary embodiments are possible without undue experimentation. All such variations and modifications are within the scope of the current teachings.

What is claimed is:

1. A method for ligating polynucleotides comprising,
providing a target polynucleotide sequence, a heat-activatable ligase, a first probe, a second probe, and a decontamination agent, thereby forming a reaction mixture,
performing a decontamimation reaction wherein the decontamination agent is substantially active at a first temperature, wherein the ligase is substantially inactive at the first temperature,
increasing the reaction temperature to a second temperature thereby increasing the activity of the ligase, and,
ligating the first probe to the second probe.

2. The method according to claim 1 wherein the decontamination agent is a uracil-N-glycosylase, and the decontamination reaction results in the removal of contaminating reaction components.

3. The method according to claim 2 wherein the uracil-N-glycosylase is least one of Arthrobacter, Micrococcus, E. coli, and combinations thereof.

4. The method according to claim 2 wherein the contaminating reaction components are carryover products from a previously performed amplification reaction.

5. The method according to claim 1 comprising a multiplexed ligation reaction wherein between 2-24 target polynucleotide sequences are queried with their corresponding first and second probes.

6. The method according to claim 1 comprising a multiplexed ligation reaction wherein between 24-96 target polynucleotide sequences are queried with their corresponding first and second probes.

7. The method according to claim 1 comprising a multiplexed ligation reaction, wherein a probe set queries a single nucleotide polymorphism, wherein the probe set comprises a first probe one and a first probe two, wherein the first probe one and first probe two distinguish between alternate alleles of the single nucleotide polymorphism.

8. The method according to claim 1 wherein the heat-activatable ligase is at least one of Afu, T4 ligase, E. coli ligase, AK16D ligase, Pfu ligase, and combinations thereof.

9. The method according to claim 1 wherein the heat-activatable ligase is chemically modified to confer substantial inactivity at the first temperature.

10. The method according to claim 1 wherein the heat-activatable ligase is complexed with an antibody to confer substantial inactivity at the first temperature.

11. The method according to claim 1 wherein the heat-activatable ligase is complexed with an aptamer to confer substantial inactivity at the first temperature.

12. The method according to claim 1 further comprising a buffer, wherein the buffer comprises an effective amount of at least one of Desferal, PEG 8000, DTT, NAD, and combinations thereof.

13. The method according to claim 1 further comprising a phosphorylation agent, wherein the phosphorylation agent is a kinase and the phosphorylation reaction results in the phosphorylation of a probe, and wherein the decontamination agent is a uracil-N-glycosylase and the decontamination reaction results in the removal of contaminating reaction components.

14. A method for ligating polynucleotides comprising,
providing a target polynucleotide sequence, a heat-activatable ligase, a first probe, a second probe, and a phosphorylation agent, thereby forming a reaction mixture,
performing a phosphorylation reaction wherein the phosphorylation agent is substantially active at a first temperature, wherein the ligase is substantially inactive at the first temperature,
increasing the reaction temperature to a second temperature thereby increasing the activity of the ligase, and,
ligating the first probe to the second probe.

15. The method according to claim 14 wherein the phosphorylation agent is a polynucleotide kinase, and the phosphorylation reaction results in the phosphorylation of a probe.

16. The method according to claim 15 wherein the polynucleotide kinase is T4 polynucleotide kinase.

17. The method according to claim 15 wherein the phosphorylated probe comprises the 5′ end of a subsequent ligation product.

18. The method according to claim 14 comprising a multiplexed ligation reaction wherein between 2-24 target polynucleotide sequences are queried with their corresponding first and second probes.

19. The method according to claim 14 comprising a multiplexed ligation reaction wherein between 24-96 target polynucleotide sequences are queried with their corresponding first and second probes.

20. The method according to claim 14 comprising a multiplexed ligation reaction, wherein a probe set queries a single nucleotide polymorphism, wherein the probe set comprises a first probe one and a first probe two, wherein the first probe one and first probe two distinguish between alternate alleles of the single nucleotide polymorphism.

21. The method according to claim 14 wherein the heat-activatable ligase is at least one of Afu, T4 ligase, E. coli ligase, AK16D ligase, Pfu ligase, and combinations thereof.

22. The method according to claim 14 wherein the heat-activatable ligase is chemically modified to confer substantial inactivity at the first temperature.

23. The method according to claim 14 wherein the heat-activatable ligase is complexed with an antibody to confer substantial inactivity at the first temperature.

24. The method according to claim 14 wherein the heat-activatable ligase is complexed with an aptamer to confer substantial inactivity at the first temperature.

25. The method according to claim 14 further comprising a buffer, wherein the buffer comprises an effective amount of at least one of Desferal, PEG 8000, DTT, NAD, and combinations thereof.

26. A reaction mixture comprising a heat-activatable ligase, a phosphorylation agent, a decontamination agent, a target polynucleotide, a first probe, and a second probe.

27. The reaction mixture according to claim 26 wherein the phosphorylation agent is a kinase.

28. The reaction mixture according to claim 27 wherein the kinase is T4 polynucleotide kinase.

29. The reaction mixture according to claim 26 wherein the decontamination agent is a uracil-N-glycosylase.

30. The reaction mixture according to claim 29 wherein the uracil-N-glycosylase is at least one of Arthrobacter, Micrococcus, E. coli, and combinations thereof.

31. The reaction mixture according to claim 26 wherein the heat-activatable ligase is at least one of Afu, T4 ligase, E. coli ligase, AK16D ligase, Pfu ligase, and combinations thereof.

32. A kit comprising a ligation master mix and at least one probe set, wherein the ligation master mix comprises at least one heat-activatable ligase, at least one phosphorylation agent, at least one decontamination agent, and at least one buffer.

33. The kit according to claim 32 further comprising at least one linker set.

34. The according to claim 32 wherein the phosphorylation agent is a kinase.

35. The kit according to claim 34 wherein the kinase is T4 polynucleotide kinase.

36. The kit according to claim 32 wherein the decontamination agent is a uracil-N-glycosylase.

37. The kit according to claim 36 wherein the uracil-N-glycosylase is at least one of Arthrobacter, Micrococcus, E. coli, and combinations thereof.

38. The kit according to claim 32 wherein the heat-activatable ligase is at least one of Afu, T4 ligase, E. coli ligase, AK16D ligase, Pfu ligase, and combinations thereof.

39. A method for reducing the number of workflow steps in a ligation reaction comprising,
providing a target polynucleotide sequence, a heat-activatable ligase, a first probe, a second probe, a phosphorylation agent, and a decontamination agent, thereby forming a reaction mixture,
performing a phosphorylation reaction comprising the phosphorylation agent at a first temperature and performing a decontamination reaction comprising the decontamination agent at the first temperature,
wherein the ligase is substantially inactive at the first temperature,
increasing the reaction temperature to a second temperature thereby increasing the activity of the ligase,
performing a ligation reaction wherein the first probe is ligated to the second probe,
thereby reducing the number of processing steps in a ligation reaction as compared with a ligation reaction in which the phosphorylation reaction and decontamination reaction are performed in reactions separate from the ligation reaction.

40. A method for ligating polynucleotides comprising,
providing a target polynucleotide sequence, a ligase, a first probe, a second probe, a decontamination agent, and a phosphorylation agent, thereby forming a reaction mixture, and,
decontaminating, phosphorylating, and ligating concurrently in the reaction mixture.
